(19) United States
(12) Patent Application Publication
(10) Pub. No.: US 2018/0271813 A1
(43) Pub. Date: Sep. 27, 2018

(54) SPRAYABLE ANALGESIC COMPOSITIONS

(71) Applicant: Achelios Therapeutics, Inc., Chapel Hill, NC (US)

(72) Inventors: Servet Buyuktimkin, San Diego, CA (US); Nadir Buyuktimkin, San Diego, CA (US); James L. Yeager, Lake Forest, IL (US)

(21) Appl. No.: 15/524,455

(22) PCT Filed: Nov. 4, 2015
(86) PCT No.: PCT/US2015/058927

§ 371 Date: May 4, 2017

Related U.S. Application Data
(60) Provisional application No. 62/077,581, filed on Nov. 10, 2014.

(51) Int. Cl. A61K 31/192; A61K 47/14; A61K 47/12; A61K 47/10; A61K 9/08; A61K 9/70

(52) U.S. Cl. CPC A61K 31/192; A61K 47/14; A61K 9/7015; A61K 47/10; A61K 9/08; A61K 47/12

(57) ABSTRACT

A sprayable analgesic composition is an aqueous ethanolic solution which contains a nonsteroidal anti-inflammatory drug (NSAID), lauryl lactate, lactic acid, glyceryl monolaurate, propylene glycol and optionally an alkoxylated alcohol.

### CROSS-REFERENCE TO RELATED APPLICATION

This application claims the benefit of U.S. Provisional Application No. 62/077,581, filed on Nov. 10, 2014, which is incorporated herein by reference in its entirety.

### FIELD OF INVENTION

This invention relates to analgesic compositions. More particularly, this invention relates to sprayable compositions containing nonsteroidal anti-inflammatory drugs.

### BACKGROUND OF THE INVENTION

Nonsteroidal anti-inflammatory drugs (NSAIDs) are known medications with analgesic and antipyretic effects. NSAIDs are used to treat pain and discomfort such as muscle strain/sprain, fever, inflammation such as rheumatoid arthritis, joint pain, and the like.

The present invention provides topical dosage forms of NSAIDs that can be applied as a spray or as an aerosol.

### SUMMARY OF INVENTION

A sprayable analgesic preparation contains a nonsteroidal anti-inflammatory drug (NSAID) together with lauryl lactate, lactic acid, and glyceryl monolaurate dissolved in a mixture of water and ethanol. The obtained aqueous ethanolic solution is useful for extended pain relief.

Optionally, the aqueous ethanolic solution can contain propylene glycol, a non-ionic surfactant having a HLB value of at least 12, and a thickener such as cellulose ethers, cross-linked alkyl acrylates, and the like.

Preferred NSAID's are the propionic acid derivatives ketoprofen, ibuprofen and naproxen, as well as the acetic acid derivatives diclofenac, indomethacin and etodolac.

### BRIEF DESCRIPTION OF DRAWINGS

In the drawings,

FIG. 1 is a bar graph showing skin permeation by ketoprofen, applied as spray compositions, at selected time intervals after application, and comparison with a commercially available, ketoprofen-containing topical gel (Profenid® Sanofi-Aventis; 2.5% ketoprofen);

FIG. 2 is a bar graph showing skin permeation by naproxen, applied as spray compositions, at selected time intervals after application, and comparison with a commercially available naproxen-containing topical gel (Naprosyn®, Syntex, 10% naproxen free acid);

FIG. 3 is a bar graph showing skin permeation by ibuprofen, applied as a spray composition, at selected time intervals after application, and comparison with a commercially available ibuprofen-containing topical gel (Ibuleve®, DDD ltd., 5% ibuprofen);

FIG. 4 is a bar graph showing skin permeation by diclofenac, and pharmaceutically acceptable salts thereof, applied as spray compositions, and comparison with a commercially available diclofenac sodium gel (Swiss Relief™, Mika Pharma GmbH, Fug, Switzerland, 4% diclofenac sodium);

FIG. 5 is a bar graph showing the effect of propylene glycol on skin permeation in ibuprofen-containing spray compositions, and comparison with a commercially available ibuprofen-containing topical gel (Ibuleve®, DDD ltd., 5% ibuprofen);

FIG. 6 is a bar graph showing skin permeation by ketoprofen with Brij 58 as a permeation enhancer, applied as spray compositions, at selected time intervals after application, and comparison with a commercially available, ketoprofen-containing topical gel (Profenid®, Sanofi-Aventis; 2.5% ketoprofen);

FIG. 7 is a bar graph showing skin permeation by ketoprofen with propylene glycol laurate as a permeation enhancer, applied as spray compositions, at selected time intervals after application, and comparison with a commercially available, ketoprofen-containing topical gel (Profenid®, Sanofi-Aventis; 2.5% ketoprofen);

FIG. 8 is a bar graph showing skin permeation by ketoprofen with propylene glycol caprylate as a permeation enhancer, applied as spray compositions, at selected time intervals after application, and comparison with a commercially available, ketoprofen-containing topical gel (Profenid®, Sanofi-Aventis; 2.5% ketoprofen);

FIG. 9 is a bar graph showing skin permeation by ketoprofen with Sorbitan monolaurate as a permeation enhancer, applied as spray compositions, at selected time intervals after application, and comparison with a commercially available, ketoprofen-containing topical gel (Profenid®, Sanofi-Aventis; 2.5% ketoprofen);

FIG. 10 is a bar graph showing skin permeation by ketoprofen with various Brij derivatives as a permeation enhancer, applied as spray compositions, at selected time intervals after application, and comparison with a commercially available, ketoprofen-containing topical gel (Profenid®, Sanofi-Aventis; 2.5% ketoprofen);

FIG. 11 is a bar graph showing skin permeation by ketoprofen, applied as a spray composition after 3 months stored at 25° C. and 40° C., at selected time intervals after application, and comparison with a commercially available, ketoprofen-containing topical gel (Profenid®, Sanofi-Aventis; 2.5% ketoprofen);

FIG. 12 is a bar graph showing skin permeation by naproxen using 5% Naproxen sodium salt and Brij 58, applied as spray compositions, at selected time intervals after application, and comparison with a commercially available naproxen-containing topical gel (Naprosyn®, Syntex, 10% naproxen free acid);

FIG. 13 is a bar graph showing skin permeation by naproxen using 2.5% Naproxen sodium salt and Brij 58, applied as spray compositions, at selected time intervals after application, and comparison with a commercially available naproxen-containing topical gel (Naprosyn®, Syntex, 10% naproxen free acid);

FIG. 14 is a bar graph showing skin permeation by ibuprofen with and without isopropyl myristate (IPM), applied as spray compositions, at selected time intervals after application, and comparison with a commercially available ibuprofen-containing topical gel (Ibuleve®, DDD ltd., 5% ibuprofen);

FIG. 15 is a bar graph showing skin permeation by ibuprofen with isopropyl myristate (IPM) and Brij 58, applied as spray compositions, at selected time intervals after application, and comparison with a commercially available ibuprofen-containing topical gel (Ibuleve®, DDD ltd., 5% ibuprofen);

FIG. 16 is a bar graph showing skin permeation by diclofenac, and pharmaceutically acceptable salts thereof, applied as cream compositions, and comparison with a commercially available diclofenac sodium gel (Voltaren®, Novartis Pharma Productions GmbH, Wehr, Germany, 1% diclofenac sodium);

FIG. 17 is a bar graph showing skin permeation by diclofenac, and pharmaceutically acceptable salts thereof, applied as spray compositions, and comparison with a commercially available diclofenac sodium gel (Swiss Relief™, Mika Pharma GmbH, Fug, Switzerland, 4% diclofenac sodium);

FIG. 18 is a bar graph showing skin permeation by diclofenac sodium with Brij 58, and different levels of propylene glycol and lactic acid, applied as spray compositions, and comparison with a commercially available diclofenac sodium gel (Voltaren®, Novartis Pharma Productions GmbH, Wehr, Germany, 1% diclofenac sodium);

FIG. 19 is a bar graph showing skin permeation by diclofenac diethylamine with Brij 58, and different levels of propylene glycol and lactic acid, applied as spray compositions, and comparison with a commercially available diclofenac sodium gel (Voltaren®, Novartis Pharma Productions GmbH, Wehr, Germany, 1% diclofenac sodium); and

FIG. 20 is a bar graph showing skin permeation by ketoprofen compositions containing different levels of propylene glycol and hydroxypropyl cellulose thickeners.

### DESCRIPTION OF PREFERRED EMBODIMENTS

The present topical compositions are clear, sprayable, aqueous ethanolic solutions that contain dissolved therein a nonsteroidal anti-inflammatory drug (NSAID). Preferred NSAIDs are the acetic acid derivatives such as indomethacin, sulindac, etodolac, tolmetin, ketorolac, nabumetone, diclofenac, and the like, including the pharmaceutically acceptable salts thereof, as well as the propionic acid derivatives such as ibuprofen, naproxen, fenoprofen, ketoprofen, flurbiprofen, oxaprozin, and the like, including the pharmaceutically acceptable salts thereof.

Illustrative NSAID salts suitable for use as active ingredients in the spray compositions are pharmaceutically acceptable salts of the aforementioned acetic acid derivatives, e.g., indomethacin salts such as indomethacin sodium, indomethacin meglumine, and the like, tolmetin salts such as tolmetin sodium, and the like, ketorolac salts such as ketorolac tromethamine, and the like, diclofenac salts such as diclofenac sodium, diclofenac diethylamine, diclofenac epolamine, and the like, as well as pharmaceutically acceptable salts of the aforementioned propionic acid derivatives, e.g., ibuprofen salts such as ibuprofen lysine, ibuprofen methylglucamine, and the like, naproxen salts such as naproxen piperazine, naproxen sodium, and the like, fenoprofen salts such as fenoprofen calcium, and the like.

Also suitable are NSAIDs such as aspirin, the enolic acid derivatives such as pizoxicam, meloxicam, tenoxicam, and the like, the fenamic acid derivatives such as mefenamic acid, meclofenamic acid, flufenamic acid, and the like, and selective COX-2 inhibitors such as celecoxib and the like, including the pharmaceutically acceptable salts thereof.

The aqueous ethanolic solutions preferably contain the NSAID in an amount in the range of about 1 to about 10 percent by weight preferably about 5 percent by weight, based on the total weight of the solution.

Also present in the solutions is lauryl lactate (C₁₅H₃₀O₃), the ester of lauryl alcohol and lactic acid having the formula

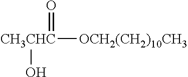

Preferably, lauryl lactate is present in the solution in an amount in the range of about 1 to about 5 weight percent, more preferably about 3 weight percent, based on the total weight of the solution.

The aqueous ethanolic solution also contains lactic acid (C₃H₆O₃; 2-hydroxypropanoic acid), preferably in an amount in the range of about 0.5 to about 5 weight percent, more preferably about 1.5 weight percent, based on the total weight of the solution; glyceryl monolaurate (C₁₅H₃₀O₄; dodecanoic acid 2,3-dihydroxypropyl ester), preferably in an amount in the range of about 2 to about 5 weight percent, more preferably about 3 weight percent, based on the total weight of the solution. Optionally, propylene glycol (C₃H₈O_{2;} propane-1,2-diol) can be present, preferably in an amount in the range of about 5 to about 30 weight percent, more preferably about 10 weight percent, based on the total weight of the solution.

The remainder of the solution is constituted by water and ethanol, preferably present in a respective weight ratio of about 0.3:1 to about 2.6:1, more preferably in a respective weight ratio of about 1:1.

The aqueous ethanolic solution can also contain, as an optional ingredient, a non-ionic surfactant having a Hydrophile-Lipophile balance (HLB) value of at least 12. Preferred non-ionic surfactants are the alkoxylated alcohols. Particularly preferred is polyethylene glycol ether of cetyl alcohol represented by the formula CH₃(CH₂)₁₄CH₂(OCH₂CH₂)ₙOH, where n has an average of 10, and having a HLB value of about 15.7.

The foregoing clear aqueous ethanolic solutions are prepared by first combining the NSAID with lauryl lactate, lactic acid, and glyceryl monolaurate and thereafter dissolving the obtained admixture by gradual addition, at ambient temperature, of propylene glycol followed by the addition of alternating aliquots of water and ethanol. The non-ionic surfactant, if desired, is added to the admixture prior to the addition of water and ethanol.

Skin permeation studies of illustrative compositions embodying the invention were performed using dermatoned human female (46 years old) cadaver skin pieces from the back (Science Care, Aurora, Colo.; 250 Micrometers thick) Franz cells (3.65 ml volume, 0.55 cm² surface area) at 35° C. using heating/stirring blocks. Receptor compartment contained saline with sodium azide (pH 7.4). Two or three replicates (25 μl and control 25 mg) were made for each solution. Sampling volume was 300 μl. Fresh buffer was replaced after each sample removal. Sampling was carried out at 2, 4, 6 and 24 hours. The samples were assayed using high performance liquid chromatography (HPLC).

Respective controls were NSAID containing gels: Profenid® gel (2.5% ketoprofen; Sanofi Aventis, France), Ibuleve® gel (5% ibuprofen; DDD Ltd., UK), Naprosyn® gel (10% naproxen free acid; Syntex, Turkey), Swiss Relief™ Spray Gel (4% diclofenac sodium) Mika Pharma GmbH, Switzerland), and Voltaren® gel (1% diclofenac sodium) Novartis Pharma Productions GmbH, Wehr, Germany.

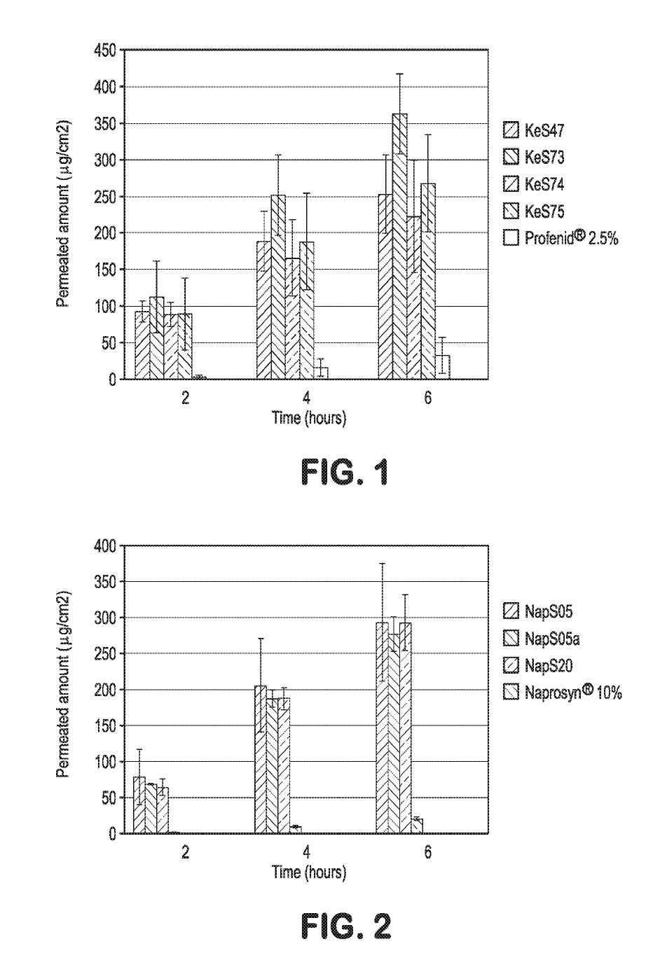

The experimental results obtained with a ketoprofen spray composition are presented in Tables 1 and 2 below, and in FIG. 1.

TABLE 1
Ketoprofen Spray Composition

| | Composition, wt. % | | | |
|---|---|---|---|---|
| Ingredients | KeS47 | KeS73 | KeS74 | KeS75 |
| Ketoprofen | 5 | 5 | 5 | 5 |
| Propylene glycol | 10 | 10 | 10 | 10 |
| Lauryl lactate | 3 | 3 | 3 | 3 |
| Lactic acid | 1.5 | 1.5 | 1.5 | 1.5 |
| Ceteth-20¹ | | 3 | | |
| Imwitor 412² | | | 3 | |
| Capmul PG-8³ | | | | 3 |
| Glyceryl monolaurate | 3 | 3 | 3 | 3 |
| Ethanol | 37.5 | 27.25 | 39.5 | 34.5 |
| Water | 40 | 47.25 | 35 | 40 |
| TOTAL | 100 | 100 | 100 | 100 |

¹CH₃(CH₂)₁₄CH₂(OCH₂CH₂)ₙOH, n average value 20; HLB 15.7; also Brij 58
²Propylene glycol laurate, HLB 4-5
³Propylene glycol monocaprylate, HLB 5-6

TABLE 2
Permeation Data

| | Cumulative Amount in Receptor, μg/cm² | | | | | | | | | |
|---|---|---|---|---|---|---|---|---|---|---|
| Time, | | | | | | | | | Profenid | |
| hours | KeS47 | ±SD | KeS73 | ±SD | KeS74 | ±SD | KeS75 | ±SD | 2.5% | ±SD |
| 2 | 92.35 | 14.49 | 112.54 | 49.36 | 88.47 | 16.40 | 88.95 | 49.36 | 2.93 | 5.93 |
| 4 | 189.43 | 40.72 | 252.20 | 54.87 | 166.05 | 52.11 | 188.46 | 65.71 | 15.74 | 21.11 |
| 6 | 253.32 | 53.34 | 363.38 | 54.64 | 222.65 | 77.00 | 267.87 | 66.92 | 32.57 | 33.53 |

The above data show that spray compositions provided better skin permeation for ketoprofen than a ketoprofen containing gel composition, and that skin permeation could be further enhanced by an alkoxylated alcohol having an HLB value of 15.7.

The experimental results obtained with a naproxen spray composition are presented in Table 3 and 4 below, and in FIGS. 2.

TABLE 3
Naproxen Spray Composition

| | Composition, wt. % | | | |
|---|---|---|---|---|
| Ingredients | NapS05 | NapS05a | NapS20 | Naprosyn ® |
| Naproxen | | | | 10 |
| Naproxen Na | 5 | 4.7 | 5 | |
| Propylene glycol | 10 | 9.5 | 10 | |
| Lauryl lactate | 3 | 2.8 | 3 | |
| Isopropyl myristate | | | 3 | |
| Lactic acid | 1.5 | 1.4 | 1.5 | |
| Ceteth-20¹ | | 2.8 | 3 | |
| Glyceryl monolaurate | 3 | 2.8 | 3 | |
| Ethanol | 55.5 | 42.7 | 41.5 | |
| Water | 22 | 33.2 | 30 | |
| TOTAL | 100 | 100 | 100 | |

TABLE 4
Naproxen Permeation Data

| Time, | Cumulative Amount in Receptor, μg/cm² | | | | | | | |
|---|---|---|---|---|---|---|---|---|
| hours | NapS05 | ±SD | NapS05a | ±SD | NapS20 | ±SD | Naprosyn ® | ±SD |
| 2 | 78.92 | 37.94 | 68.91 | 0.54 | 64.42 | 11.20 | 1.65 | 0.28 |
| 4 | 206.15 | 65.01 | 187.63 | 11.82 | 187.47 | 15.16 | 9.67 | 1.17 |
| 6 | 293.37 | 81.72 | 277.23 | 23.83 | 292.88 | 38.90 | 20.38 | 2.42 |

The above data show that naproxen containing spray compositions provided better skin permeation for naproxen than the Naprosyn® 10% naproxen gel. The incorporation of higher levels of water did not reduce the permeation of naproxen.

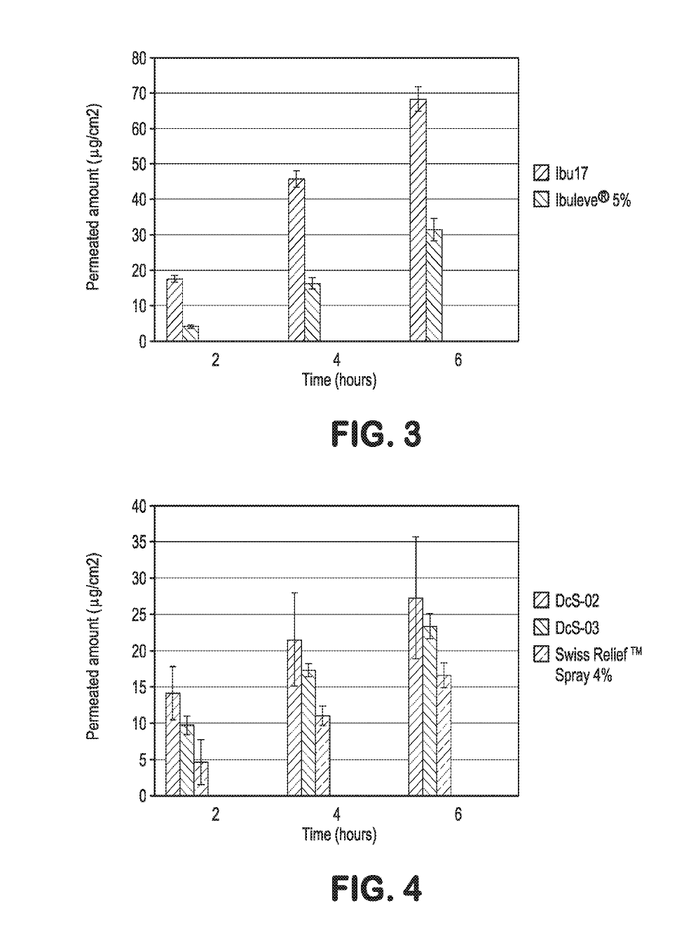

The experimental results obtained with an ibuprofen spray composition are presented in Tables 5 and 6 below, and in FIG. 3. The experimental procedure was the same as that for the ketoprofen and naproxen spray compositions, except that the dermatomed cadaver skin was that of a human male, 72 years old.

TABLE 5
Ibuprofen Spray Composition

Composition, wt. %

| Ingredients | Ibu17 | Ibuleve ® |
|---|---|---|
| Ibuprofen | 5 | 5 |
| Lauryl lactate | 3 | |
| Lactic acid | 1.5 | |
| Glyceryl monolaurate | 3 | |
| Ethanol | 47.5 | |
| Water | 40 | |
| TOTAL | 100 | |

TABLE 6
Ibuprofen Permeation Data

| | |
|---|---|
| Time, | Cumulative Amount in Receptor, μg/cm² |

| hours | Ibu17 | ±SD | Ibuleve ® | ±SD |
|---|---|---|---|---|
| 2 | 17.59 | 0.96 | 4.12 | 1.22 |
| 4 | 45.81 | 0.75 | 16.31 | 0.45 |
| 6 | 68.39 | 0.16 | 31.47 | 0.32 |

The above data show that an ibuprofen containing spray composition provided better skin permeation for ibuprofen than the Ibuleve® ibuprofen gel.

The experimental results obtained with a diclofenac spray composition are presented in Tables 7 and 8 below, and in FIG. 4. The experimental procedure was the same as that for the ketoprofen and naproxen spray compositions except that the dermatomed cadaver skin was that of a 79-year old human male.

TABLE 7
Diclofenac Spray Composition

Composition, wt. %

| Ingredients | DcS02 | DcS03 | Swiss Relief ™ |
|---|---|---|---|
| Diclofenac Na | 1 | | 4⁴ |
| Diclofenac diethylamine | | 1 | |
| Propylene glycol | 10 | 10 | |
| Lauryl lactate | 3 | 3 | |
| Lactic acid | 1.5 | 1.5 | |
| Glyceryl monolaurate | 3 | 3 | |
| Ethanol | 48.5 | 48.5 | |
| Water | 33 | 33 | |
| TOTAL | 100 | 100 | |

⁴Swiss Relief ™ spray gel contains 4 wt. % diclofenac sodium together with inactive ingredients isopropyl alcohol, soy bean lecithin, ethanol, disodium phosphate dodecahydrate, sodium dihodrogen phosphate dehydrate, sodium edetate, propylene glycol, peppermint oil, ascorbyl palmitate, hydrochloric acid (10% w/w), sodium hydroxide (10% w/w), purified water.

TABLE 8
Diclofenac Permeation Data

| | Cumulative Amount in Receptor, μg/cm² | | | | | |
|---|---|---|---|---|---|---|
| Time, | | | | | Swiss Relief ™ | |
| hours | DcS-02 | ±SD | DcS-03 | ±SD | spray gel | ±SD |
| 2 | 14.16 | 3.67 | 9.71 | 1.25 | 4.64 | 3.11 |
| 4 | 21.51 | 6.42 | 17.30 | 0.93 | 11.08 | 1.33 |
| 6 | 27.31 | 8.43 | 23.39 | 1.76 | 16.62 | 1.70 |

The above data show that a diclofenac containing spray composition provided better skin permeation for diclofenac than a spray gel composition that has a relatively larger concentration of diclofenac.

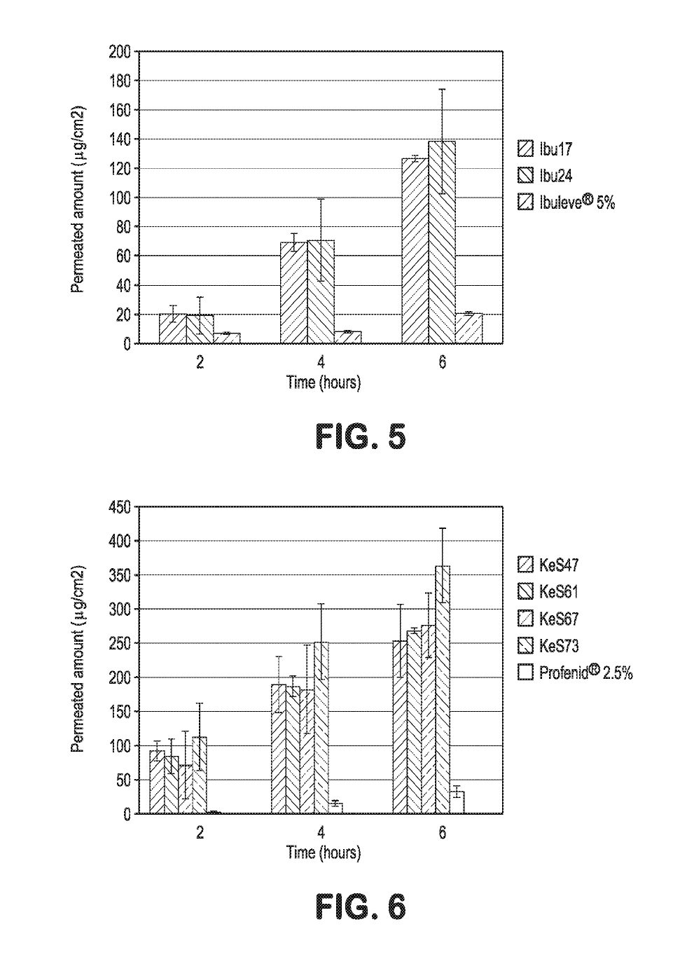

The effect of propylene glycol in an ibuprofen spray composition was investigated using cadaver skin from a 72 year-old human male. The experimental results are presented in Tables 9 and 10 below, and in FIG. 5.

TABLE 9
Ibuprofen Spray Compositions

| | Composition, wt. % | | |
|---|---|---|---|
| Ingredients | Ibu17 | Ibu24 | Ibuleve ® |
| Ibuprofen | 5 | 5 | 5 |
| Propylene glycol | | 10 | |
| Lauryl lactate | 3 | 3 | |
| Lactic acid | 1.5 | 1.5 | |
| Glyceryl monolaurate | 3 | 3 | |
| Ethanol | 47.5 | 37.5 | |
| Water | 40 | 40 | |
| TOTAL | 100 | 100 | |

TABLE 10
Ibuprofen Permeation Data

| Time, | Cumulative Amount in Receptor, μg/cm² | | | | | |
|---|---|---|---|---|---|---|
| hours | Ibu17 | ±SD | Ibu24 | ±SD | Ibuleve ® | ±SD |
| 2 | 20.22 | 5.59 | 19.36 | 12.52 | 7.17 | 12.42 |
| 4 | 69.45 | 5.95 | 71.08 | 27.82 | 8.65 | 1.80 |
| 6 | 127.06 | 2.01 | 138.70 | 35.68 | 20.97 | 8.20 |

The above data show that propylene glycol in the spray composition enhanced the skin penetration of ibuprofen.

The experimental results obtained with a ketoprofen spray composition and a nonionic surfactant, polyoxyethylene (20) cetyl ether (Brij 58), as a permeation enhancer are presented in Tables 11 and 12 below, and in FIG. 6.

TABLE 11
Ketoprofen and a Nonionic Surfactant Spray Composition

| | Composition, wt. % | | | | |
|---|---|---|---|---|---|
| Ingredients | KeS47 | KeS61 | KeS67 | KeS73 | KeS73a |
| Ketoprofen | 5 | 5 | 5 | 5 | 5 |
| Propylene glycol | 10 | 10 | 10 | 10 | 10 |
| Lauryl lactate | 3 | 3 | 0 | 3 | 3 |
| Lactic acid | 1.5 | 1.5 | 1.5 | 1.5 | 1.5 |
| Brij 58¹ | | 3 | 3 | 3 | 3 |
| Glyceryl monolaurate | 3 | 0 | 3 | 3 | 3 |
| Ethanol | 37.5 | 37.5 | 37.5 | 27.25 | 25.25 |
| Water | 40 | 40 | 40 | 47.25 | 49.25 |
| TOTAL | 100 | 100 | 100 | 100 | 100 |

TABLE 12
Permeation Data

| | Cumulative Amount in Receptor, μg/cm² | | | | | | | | | |
|---|---|---|---|---|---|---|---|---|---|---|
| Time, | | | | | | | | | Profenid | |
| hours | KeS47 | ±SD | KeS61 | ±SD | KeS67 | ±SD | KeS73 | ±SD | 2.5% | ±SD |
| 2 | 92.35 | 14.49 | 84.55 | 25.06 | 71.71 | 49.49 | 112.54 | 49.36 | 15.60 | 5.93 |
| 4 | 189.43 | 40.72 | 186.97 | 15.02 | 182.54 | 64.55 | 252.20 | 54.87 | 70.75 | 21.11 |
| 6 | 253.32 | 53.34 | 267.88 | 3.95 | 275.99 | 47.09 | 363.38 | 54.64 | 134.62 | 33.53 |

The above data show that the addition of Brij 58 helped to increase water levels. All formulations exhibited similar permeation behavior; however, KeS73 showed slightly higher permeation. KeS73a was slightly cloudy.

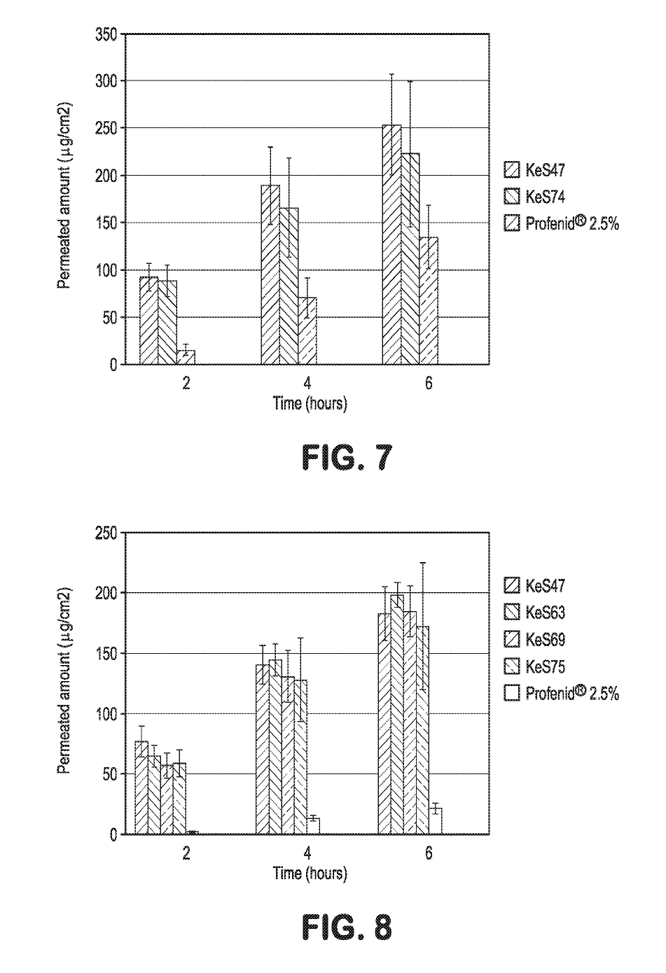

The experimental results obtained with a ketoprofen spray composition and propylene glycol laurate as a permeation enhancer are presented in Tables 13 and 14 below, and in FIG. 7.

TABLE 13
Ketoprofen and Propylene Glycol Laurate Spray Composition

| | Composition, wt. % | | | |
|---|---|---|---|---|
| Ingredients | KeS47 | KeS62 | KeS68 | KeS74 |
| Ketoprofen | 5 | 5 | 5 | 5 |
| Propylene glycol | 10 | 10 | 10 | 10 |
| Lauryl lactate | 3 | 3 | 0 | 3 |
| Lactic acid | 1.5 | 1.5 | 1.5 | 1.5 |
| Propylene glycol laurate | | 3 | 3 | 3 |
| Glyceryl monolaurate | 3 | 0 | 3 | 3 |
| Ethanol | 37.5 | 42.5 | 42.5 | 39.5 |
| Water | 40 | 35 | 35 | 35 |
| TOTAL | 100 | 100 | 100 | 100 |

TABLE 14
Permeation Data

| | Cumulative Amount in Receptor, μg/cm² | | | | | |
|---|---|---|---|---|---|---|
| Time, | | | | | Profenid | |
| hours | KeS47 | ±SD | KeS74 | ±SD | 2.5% | ±SD |
| 2 | 92.35 | 14.49 | 88.47 | 16.40 | 15.60 | 5.93 |
| 4 | 189.43 | 40.72 | 166.05 | 52.11 | 70.75 | 21.11 |
| 6 | 253.32 | 53.34 | 222.65 | 77.00 | 134.62 | 33.53 |

Only formulations KeS47 and KeS74 gave clear solutions. The above data show that the KeS47 and KeS74 formulations exhibited nearly the same permeation behavior.

The experimental results obtained with a ketoprofen spray composition and propylene glycol caprylate as a permeation enhancer are presented in Tables 15 and 16 below, and in FIG. 8. The experimental procedure was the same as that for the previous ketoprofen spray compositions, except that the dermatomed cadaver skin was that of a human male, 79 years old.

TABLE 15
Ketoprofen and Propylene Glycol Caprylate Spray Composition

| | Composition, wt. % | | | |
|---|---|---|---|---|
| Ingredients | KeS47 | KeS63 | KeS69 | KeS75 |
| Ketoprofen | 5 | 5 | 5 | 5 |
| Propylene glycol | 10 | 10 | 10 | 10 |
| Lauryl lactate | 3 | 3 | 0 | 3 |
| Lactic acid | 1.5 | 1.5 | 1.5 | 1.5 |
| Propylene glycol caprylate | | 3 | 3 | 3 |
| Glyceryl monolaurate | 3 | 0 | 3 | 3 |
| Ethanol | 37.5 | 37.5 | 37.5 | 34.5 |
| Water | 40 | 40 | 40 | 40 |
| TOTAL | 100 | 100 | 100 | 100 |

TABLE 16
Permeation Data

| | Cumulative Amount in Receptor, μg/cm² | | | | | | | | | |
|---|---|---|---|---|---|---|---|---|---|---|
| Time, | | | | | | | | | Profenid | |
| hours | KeS47 | ±SD | KeS63 | ±SD | KeS69 | ±SD | KeS75 | ±SD | 2.5% | ±SD |
| 2 | 77.12 | 12.81 | 64.90 | 9.02 | 57.22 | 10.33 | 58.98 | 10.81 | 2.27 | 0.44 |
| 4 | 140.68 | 16.03 | 144.77 | 13.15 | 131.23 | 21.40 | 128.25 | 34.31 | 13.60 | 2.05 |
| 6 | 183.17 | 22.46 | 198.59 | 10.20 | 184.99 | 21.11 | 172.43 | 52.30 | 21.69 | 4.50 |

The above data show that all of the formulations exhibited comparable permeation behavior.

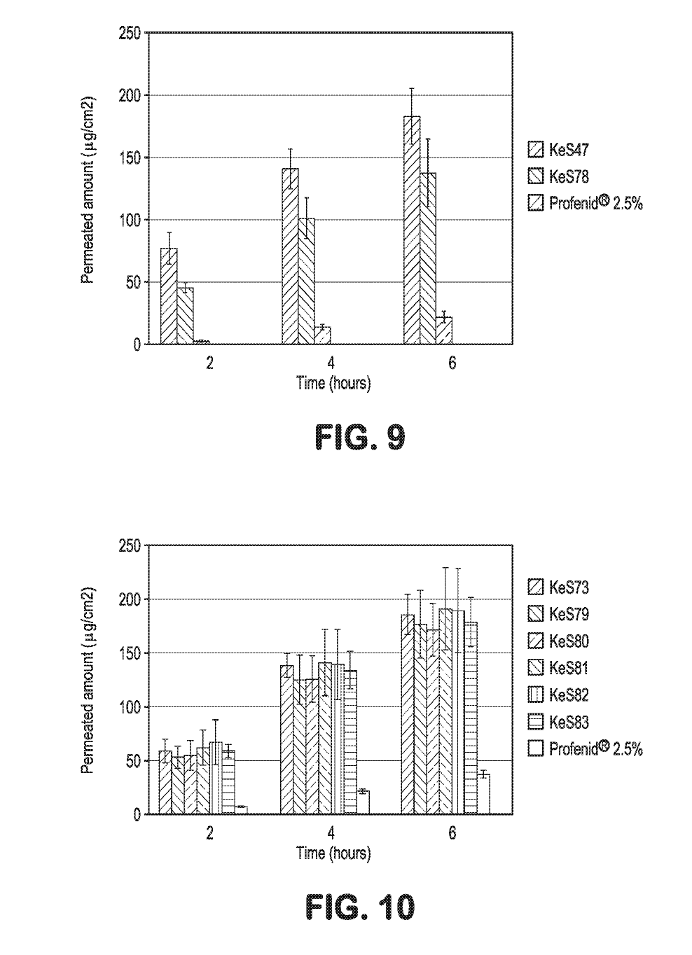

The experimental results obtained with a ketoprofen spray composition and Sorbitan monolaurate as a permeation enhancer are presented in Tables 17 and 18 below, and in FIG. 9. The experimental procedure was the same as that for the previous ketoprofen spray compositions, except that the dermatomed cadaver skin was that of a human male, 79 years old.

TABLE 17
Ketoprofen and Sorbitan Monolaurate Spray Composition

| | Composition, wt. % | | | |
|---|---|---|---|---|
| Ingredients | KeS47 | KeS66 | KeS72 | KeS78 |
| Ketoprofen | 5 | 5 | 5 | 5 |
| Propylene glycol | 10 | 10 | 10 | 10 |
| Lauryl lactate | 3 | 3 | 0 | 3 |
| Lactic acid | 1.5 | 1.5 | 1.5 | 1.5 |
| Sorbitan monolaurate | | 3 | 3 | 3 |
| Glyceryl monolaurate | 3 | 0 | 3 | 3 |
| Ethanol | 37.5 | 57.5 | 57.5 | 34.5 |
| Water | 40 | 20 | 20 | 40 |
| TOTAL | 100 | 100 | 100 | 100 |

TABLE 18
Permeation Data

| | Cumulative Amount in Receptor, μg/cm² | | | | | |
|---|---|---|---|---|---|---|
| Time, | | | | | Profenid | |
| hours | KeS47 | ±SD | KeS78 | ±SD | 2.5% | ±SD |
| 2 | 77.12 | 12.81 | 45.37 | 4.10 | 2.27 | 0.44 |
| 4 | 140.68 | 16.03 | 101.16 | 16.60 | 13.60 | 2.05 |
| 6 | 183.17 | 22.46 | 137.44 | 27.22 | 21.69 | 4.50 |

Only formulations KeS47 and KeS78 gave clear solutions. The above data show that permeation from KeS78 was slightly lower than KeS47.

The experimental results obtained with a ketoprofen spray composition and various Brij derivatives as a permeation enhancer are presented in Tables 19 and 20 below, and in FIG. 10. The experimental procedure was the same as that for the previous ketoprofen spray compositions, except that the dermatomed cadaver skin was that of a human male, 79 years old.

TABLE 19
Ketoprofen and Non-ionic Surfactant Spray Composition

| | Composition, wt. % | | | | | |
|---|---|---|---|---|---|---|
| Ingredients | KeS73 | KeS79 | KeS80 | KeS81 | KeS82 | KeS83 |
| Ketoprofen | 5 | 5 | 5 | 5 | 5 | 5 |
| Propylene | 10 | 10 | 10 | 10 | 10 | 10 |
| glycol | | | | | | |
| Lauryl lactate | 3 | 3 | 3 | 3 | 3 | 3 |
| Lactic acid | 1.5 | 1.5 | 1.5 | 1.5 | 1.5 | 1.5 |
| Brij 58¹ | 3 | | | | | |
| Brij 30⁵ | | 3 | | | | |
| Brij 35⁶ | | | 3 | | | |
| Brij 72⁷ | | | | 3 | | |
| Brij 98⁸ | | | | | 3 | |
| Brij 721⁹ | | | | | | 3 |
| Glyceryl | 3 | 3 | 3 | 3 | 3 | 3 |
| monolaurate | | | | | | |
| Ethanol | 27.25 | 27.25 | 27.25 | 27.25 | 27.25 | 27.25 |
| Water | 47.25 | 47.25 | 47.25 | 47.25 | 47.25 | 47.25 |
| TOTAL | 100 | 100 | 100 | 100 | 100 | 100 |

⁵poly(oxyethylene)(4) lauryl ether
⁶poly(oxyethylene)(23) lauryl ether
⁷poly(oxyethylene)(2) stearyl ether
⁸poly(oxyethylene)(20) oleyl ether
⁹poly(oxyethylene)(21) stearyl ether

TABLE 20
Permeation Data

| | Cumulative Amount in Receptor, μg/cm² | | | | | | | | | | | | | |
|---|---|---|---|---|---|---|---|---|---|---|---|---|---|---|
| Time, | | | | | | | | | | | | | Profenid | |
| hours | KeS73 | ±SD | KeS79 | ±SD | KeS80 | ±SD | KeS81 | ±SD | KeS82 | ±SD | KeS83 | ±SD | 2.5% | ±SD |
| 2 | 58.92 | 11.11 | 53.39 | 10.39 | 54.79 | 13.75 | 61.91 | 16.23 | 67.06 | 20.54 | 59.12 | 6.21 | 7.15 | 0.42 |
| 4 | 138.70 | 11.05 | 125.49 | 22.77 | 125.93 | 21.40 | 141.07 | 30.91 | 139.71 | 32.62 | 134.15 | 17.36 | 21.66 | 1.94 |
| 6 | 185.84 | 18.67 | 176.90 | 31.52 | 171.52 | 24.45 | 191.21 | 38.25 | 189.49 | 39.15 | 178.84 | 23.19 | 37.41 | 3.53 |

The above data show that all formulations containing the non-ionic surfactants permitted higher water content while showing similar behavior with respect to permeation.

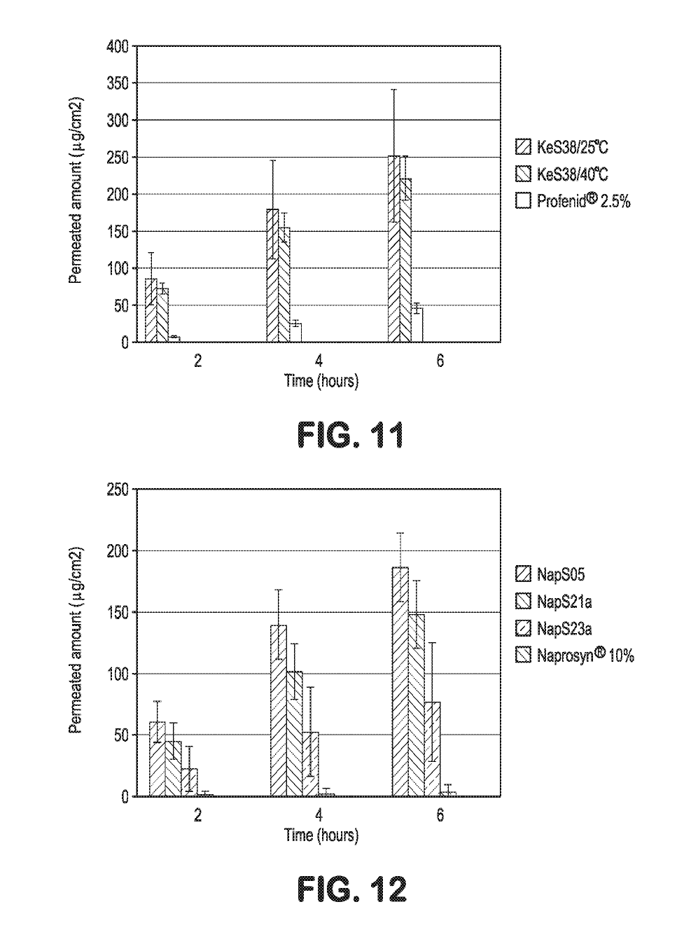

The experimental results showing permeation of a ketoprofen spray composition after storage for three (3) months at 25° C. and 40° C. is presented in Tables 21 and 22 below, and in FIG. 11. The experimental procedure was the same as that for the previous ketoprofen spray compositions, except that the dermatomed cadaver skin was from the thigh of a human male, 79 years old.

TABLE 21
Ketoprofen Spray Composition

Composition, wt. %

| Ingredients | KeS38/25° C. | KeS38/40° C. |
|---|---|---|
| Ketoprofen | 5 | 5 |
| Lauryl lactate | 3 | 3 |
| Lactic acid | 1.5 | 1.5 |
| Glyceryl monolaurate | 3 | 3 |
| Ethanol | 47.5 | 47.25 |
| Water | 40 | 40 |
| TOTAL | 100 | 100 |

TABLE 22
Permeation Data

| | Cumulative Amount in Receptor, μg/cm² | | | | | |
|---|---|---|---|---|---|---|
| Time, | | | | | Profenid | |
| hours | KeS38/25° C. | ±SD | KeS38/40° C. | ±SD | 2.5% | ±SD |
| 2 | 85.61 | 35.14 | 72.35 | 7.44 | 6.97 | 1.23 |
| 4 | 178.97 | 66.42 | 154.72 | 19.61 | 25.34 | 3.96 |
| 6 | 251.24 | 89.44 | 220.46 | 29.64 | 45.35 | 6.86 |

The above data show that both formulations exhibited similar permeation behavior after three months of storage.

The experimental results obtained with a naproxen spray composition using 5% Naproxen sodium and Brij 58 are presented in Table 23 and 24 below, and in FIG. 12. The experimental procedure was the same as that for the previous spray compositions, except that the dermatomed cadaver skin was from the thigh of a human male, 79 years old.

TABLE 23
Naproxen Spray Composition Using 5%
Naproxen Sodium and a Non-ionic Surfactant

| | Composition, wt. % | | | | |
|---|---|---|---|---|---|
| Ingredients | NapS05 | NapS21a | NapS22a | NapS23a | NapS24 |
| Naproxen | 0 | 0 | 0 | 0 | 0 |
| Naproxen Na | 5 | 5 | 5 | 5 | 5 |
| Propylene glycol | 10 | 10 | 10 | 10 | 10 |
| Lauryl lactate | 3 | 3 | 3 | 3 | 3 |
| Lactic acid | 1.5 | 0.5 | 3 | 0.5 | 3 |
| Brij 58¹ | | 0 | 0 | 3 | 3 |
| Glyceryl | 3 | 3 | 3 | 3 | 3 |
| monolaurate | | | | | |
| Ethanol | 55.5 | 34.5 | 46 | 30 | 47 |
| Water | 22 | 44 | 30 | 45.5 | 26 |
| TOTAL | 100 | 100 | 100 | 100 | 100 |

TABLE 24
Naproxen Permeation Data

| Time, | Cumulative Amount in Receptor, μg/cm² | | | | | | | |
|---|---|---|---|---|---|---|---|---|
| hours | NapS05 | ±SD | NapS21a | ±SD | NapS23a | ±SD | Naprosyn ® | ±SD |
| 2 | 60.77 | 16.57 | 44.92 | 14.84 | 22.70 | 18.23 | 1.67 | 2.90 |
| 4 | 139.90 | 28.22 | 101.66 | 22.94 | 52.91 | 36.06 | 2.37 | 4.10 |
| 6 | 186.06 | 27.93 | 147.81 | 27.39 | 77.01 | 48.09 | 3.61 | 6.25 |

The above data show that by decreasing the level of water and lactic acid, formulations with higher levels of ethanol were prepared. Reduction in lactic acid and addition of Brij 58 resulted in lower skin permeation. A precipitate was noted in NapS22a and NapS24.

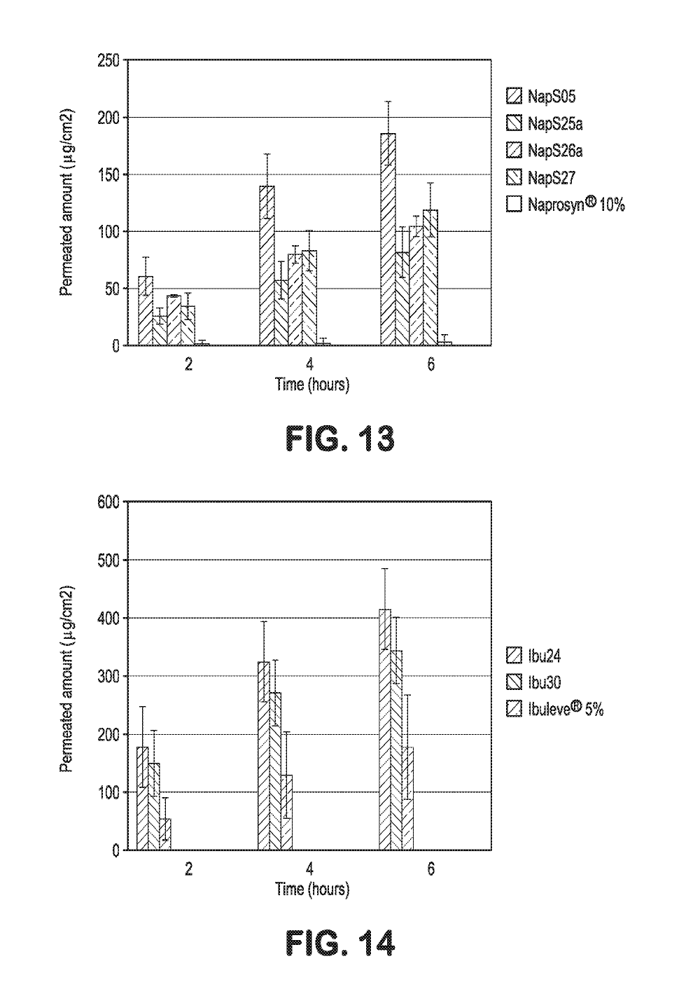

The experimental results obtained with a naproxen spray composition using 2.5% Naproxen sodium and Brij 58 are presented in Table 25 and 26 below, and in FIG. 13. The experimental procedure was the same as that for the previous spray compositions, except that the dermatomed cadaver skin was from the thigh of a human male, 79 years old.

TABLE 25
Naproxen Spray Composition Using 2.5% Naproxen Sodium and a Non-ionic Surfactant

| | Composition, wt. % | | | | | | |
|---|---|---|---|---|---|---|---|
| Ingredients | NapS05 | NapS25 | NapS25a | NapS26 | NapS26a | NapS27 | NapS28 |
| Naproxen | 0 | 0 | 0 | 0 | 0 | 0 | 0 |
| Naproxen Na | 5 | 2.5 | 2.5 | 2.5 | 2.5 | 2.5 | 2.5 |
| Propylene glycol | 10 | 10 | 10 | 10 | 10 | 10 | 10 |
| Lauryl lactate | 3 | 3 | 3 | 3 | 3 | 3 | 3 |
| Lactic acid | 1.5 | 0.5 | 0.5 | 1.5 | 1.5 | 0.5 | 1.5 |
| Brij 58¹ | | 3 | 3 | 3 | 3 | 0 | 0 |
| Glyceryl monolaurate | 3 | 3 | 3 | 3 | 3 | 3 | 3 |
| Ethanol | 55.5 | 22 | 32 | 37 | 40 | 36 | 45 |
| Water | 22 | 56 | 46 | 40 | 37 | 45 | 35 |
| TOTAL | 100 | 100 | 100 | 100 | 100 | 100 | 100 |

A precipitate was noted in NapS05, NapS25, NapS26 and NapS28.

TABLE 26
Naproxen Permeation Data

| Time, | Cumulative Amount in Receptor, μg/cm² | | | | | | | | | |
|---|---|---|---|---|---|---|---|---|---|---|
| hours | NapS05 | ±SD | NapS25a | ±SD | NapS26a | ±SD | NapS27 | ±SD | Naprosyn ® | ±SD |
| 2 | 60.77 | 16.57 | 26.05 | 7.07 | 43.55 | 1.01 | 34.57 | 11.52 | 1.67 | 2.90 |
| 4 | 139.90 | 28.22 | 57.48 | 16.40 | 79.99 | 7.85 | 83.28 | 17.65 | 2.37 | 4.10 |
| 6 | 186.06 | 27.93 | 82.19 | 21.96 | 104.69 | 8.99 | 119.11 | 23.73 | 3.61 | 6.25 |

The above data show that reduction of Naproxen levels to 2.5% caused a significant reduction in skin permeation.

The effect of isopropyl myristate in an ibuprofen spray composition was investigated. The experimental results are presented in Tables 27 and 28 below, and in FIG. 14. The experimental procedure was the same as that for the previous spray compositions, except that the dermatomed cadaver skin was from the thigh of a human male, 79 years old.

TABLE 27
Ibuprofen with and without Isopropyl Myristate Spray Compositions

Composition,
wt. %

| Ingredients | Ibu24 | Ibu30 |
|---|---|---|
| Ibuprofen | 5 | 5 |
| Propylene glycol | 10 | 10 |
| Isopropyl myristate | 0 | 3 |
| Lauryl lactate | 3 | 3 |
| Lactic acid | 1.5 | 1.5 |
| Glyceryl monolaurate | 3 | 3 |
| Ethanol | 37.5 | 39.5 |
| Water | 40 | 35 |
| TOTAL | 100 | 100 |

TABLE 28
Ibuprofen with and without Isopropyl Myristate Permeation Data

| Time, | Cumulative Amount in Receptor, μg/cm² | | | | | |
|---|---|---|---|---|---|---|
| hours | Ibu24 | ±SD | Ibu30 | ±SD | Ibuleve ® | ±SD |
| 2 | 177.88 | 34.92 | 149.67 | 43.10 | 53.86 | 36.32 |
| 4 | 324.99 | 58.07 | 271.38 | 62.99 | 129.82 | 74.30 |
| 6 | 415.42 | 62.34 | 344.02 | 61.75 | 177.62 | 89.78 |

The above data show that addition of isopropyl myristate in the spray composition did not further enhance the skin permeation of ibuprofen.

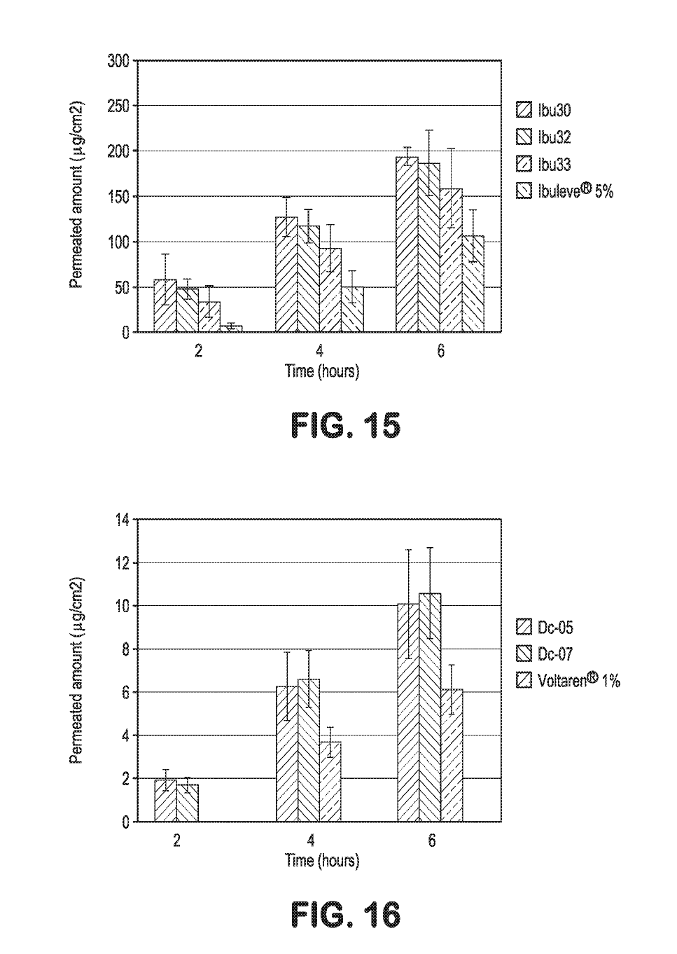

The effect of isopropyl myristate and Brij 58 in an ibuprofen spray composition was investigated. The experimental results are presented in Tables 29 and 30 below, and in FIG. 15. The experimental procedure was the same as that for the previous spray compositions, except that the dermatomed cadaver skin was from the back of a human male, 46 years old.

TABLE 29
Ibuprofen with Isopropyl Myristate and Brij 58 Spray Compositions

| | Composition, wt. % | | |
|---|---|---|---|
| Ingredients | Ibu30 | Ibu32 | Ibu33 |
| Ibuprofen | 5 | 5 | 5 |
| Propylene glycol | 10 | 10 | 10 |
| Isopropyl myristate | 3 | 0 | 3 |
| Lauryl lactate | 3 | 3 | 3 |
| Lactic acid | 1.5 | 1.5 | 1.5 |
| Brij 58¹ | | 3 | 3 |
| Glyceryl monolaurate | 3 | 3 | 3 |
| Ethanol | 39.5 | 28 | 31.5 |
| Water | 35 | 46.5 | 40 |
| TOTAL | 100 | 100 | 100 |

TABLE 30
Ibuprofen Permeation Data

| | Cumulative Amount in Receptor, μg/cm² | | | | | | | |
|---|---|---|---|---|---|---|---|---|
| Time, hours | Ibu30 | ±SD | Ibu32 | ±SD | Ibu33 | ±SD | Ibuleve ® | ±SD |
| 2 | 58.28 | 27.72 | 47.79 | 10.56 | 33.70 | 17.05 | 7.06 | 3.27 |
| 4 | 127.23 | 21.44 | 117.41 | 18.49 | 93.08 | 25.68 | 50.52 | 17.68 |
| 6 | 193.89 | 9.97 | 187.01 | 36.12 | 159.15 | 43.60 | 106.53 | 28.78 |

The above data show that addition of Brij 58 helped to increase the level of water in the formulation; however, the addition of isopropyl myristate and Brij 58 in the spray composition did not further enhance the skin permeation of ibuprofen.

The effects of two types of diclofenac were investigated. The experimental results obtained with a diclofenac cream composition using diclofenac sodium and diclofenac diethylamine are presented in Tables 31 and 32, below, and in FIG. 16. The dermatomed cadaver skin was from the back of a human male, 79 years old.

TABLE 31
Diclofenac Cream Composition

Composition, wt. %

| Ingredients | Dc-05 | Dc-07 |
|---|---|---|
| Diclofenac Na | 1 | |
| Diclofenac diethylamine | | 1 |
| Carbopol 980 NF¹⁰ | 0.5 | 0.5 |
| Ultrez 10¹¹ | 1 | 1 |
| Deionized water | 63.95 | 63.95 |
| Disodium EDTA | 0.05 | 0.05 |
| Isopropyl myristate | 3 | 3 |
| Ethanol | 10 | 10 |
| Propylene glycol | 10 | 10 |
| Isopropanol | 9 | 9 |
| Triethanolamine, NF | 1.5 | 1.5 |
| TOTAL | 100 | 100 |

¹⁰acrylic acid homopolymer
¹¹cross-linked polyacrylic acid polymer

TABLE 32
Diclofenac Permeation Data

| Time, | Cumulative Amount in Receptor, μg/cm² | | | | | |
|---|---|---|---|---|---|---|
| hours | Dc-05 | ±SD | Dc-07 | ±SD | Voltaren 1% | ±SD |
| 2 | 1.94 | 1.95 | 1.70 | 2.95 | 0.00 | 0.00 |
| 4 | 6.28 | 2.27 | 6.62 | 4.46 | 3.70 | 0.70 |
| 6 | 10.10 | 2.85 | 10.60 | 5.96 | 6.13 | 1.14 |

The above data show that skin permeation for diclofenac sodium and diclofenac diethylamine cream formulations was similar.

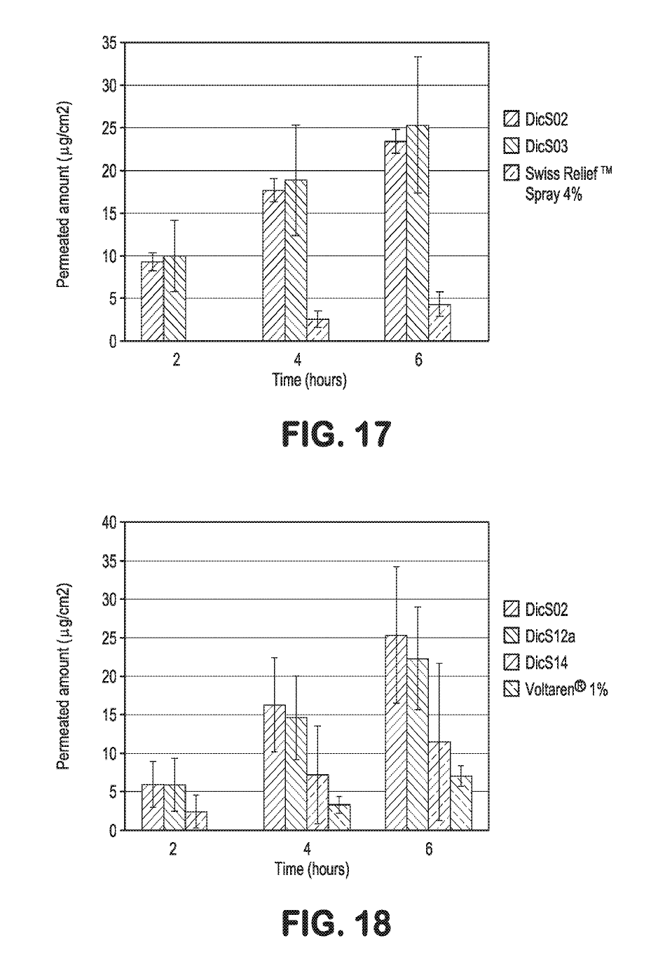

The experimental results obtained with a diclofenac spray composition using diclofenac sodium and diclofenac diethylamine are presented in Tables 33 and 34, below, and in FIG. 17. The experimental procedure was the same as that for the previous spray compositions, except that the dermatomed cadaver skin was from the back of a human male, 79 years old.

TABLE 33
Diclofenac Spray Composition

Composition, wt. %

| Ingredients | DcS02 | DcS03 |
|---|---|---|
| Diclofenac Na | 1 | |
| Diclofenac diethylamine | | 1 |
| Propylene glycol | 10 | 10 |
| Lauryl lactate | 3 | 3 |
| Lactic acid | 1.5 | 1.5 |
| Glyceryl monolaurate | 3 | 3 |
| Ethanol | 48.5 | 48.5 |
| Water | 33 | 33 |
| TOTAL | 100 | 100 |

TABLE 34
Diclofenac Permeation Data

| | Cumulative Amount in Receptor, μg/cm² | | | | | |
|---|---|---|---|---|---|---|
| Time, | | | | | Swiss Relief ™ | |
| hours | DcS-02 | ±SD | DcS-03 | ±SD | spray gel | ±SD |
| 2 | 9.29 | 1.06 | 9.98 | 4.16 | 0 | 0 |
| 4 | 17.69 | 1.30 | 18.91 | 6.47 | 2.57 | 0.93 |
| 6 | 23.43 | 1.38 | 25.33 | 7.99 | 4.31 | 1.44 |

The above data show that a diclofenac containing spray composition provided better skin permeation for diclofenac than a spray gel composition that has a relatively larger concentration of diclofenac.

The effects of propylene glycol, Brij 58 and lactic acid on diclofenac skin permeation were investigated. The experimental results obtained with a diclofenac spray composition using diclofenac sodium, Brij 58, and different levels of propylene glycol and lactic acid are presented in Tables 35 and 36, below, and in FIG. 18. The experimental procedure was the same as that for the previous spray compositions, except that the dermatomed cadaver skin was from the thigh of a human male, 79 years old.

TABLE 35
Diclofenac Spray Composition

| | Composition, wt. % | | | |
|---|---|---|---|---|
| Ingredients | DcS02 | DcS12 | DcS12a | DcS14 |
| Diclofenac Na | 1 | 1 | 1 | 1 |
| Propylene glycol | 10 | 15 | 10 | 10 |
| Lauryl lactate | 3 | 3 | 3 | 3 |
| Lactic acid | 1.5 | 1.5 | 1.5 | 0.5 |
| Brij 58¹ | | 3 | 3 | 3 |
| Glyceryl | 3 | 3 | 3 | 3 |
| monolaurate | | | | |
| Ethanol | 48.5 | 25 | 43.5 | 34.5 |
| Water | 33 | 53.5 | 35 | 45 |
| TOTAL | 100 | 100 | 100 | 100 |

Composition DcS12 was cloudy.

TABLE 36
Diclofenac Permeation Data

| | Cumulative Amount in Receptor, μg/cm² | | | | | | | |
|---|---|---|---|---|---|---|---|---|
| Time hours, | DcS02 | ±SD | DcS12a | ±SD | DcS14 | ±SD | Voltaren 1% | ±SD |
| 2 | 5.98 | 2.94 | 5.97 | 3.42 | 2.47 | 2.17 | 0.00 | 0.00 |
| 4 | 16.33 | 6.10 | 14.68 | 5.39 | 7.24 | 6.34 | 3.36 | 1.11 |
| 6 | 25.37 | 8.82 | 22.33 | 6.67 | 11.52 | 10.23 | 7.10 | 1.33 |

Addition of a higher level of propylene glycol enhanced the water content but caused formulation DcS12 to precipitate. The above data show that a diclofenac spray composition with a lower level of lactic acid showed a lower level of skin permeation of diclofenac.

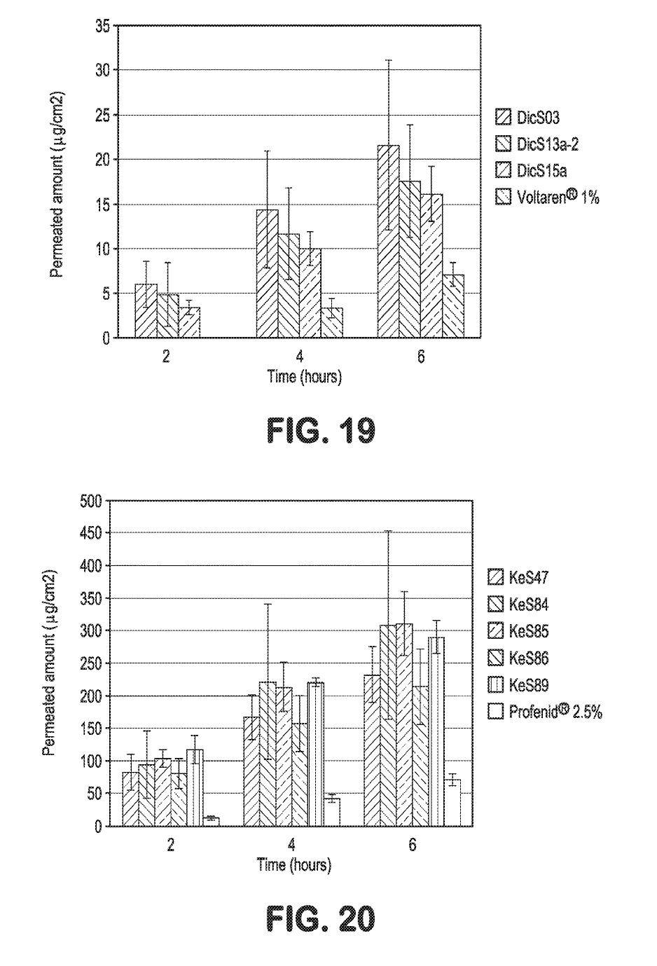

The effects of propylene glycol, Brij 58 and lactic acid on diclofenac skin permeation were investigated. The experimental results obtained with a diclofenac spray composition using diclofenac diethylamine, Brij 58, and different levels of propylene glycol and lactic acid are presented in Tables 37 and 38, below, and in FIG. 19. The experimental procedure was the same as that for the previous spray compositions, except that the dermatomed cadaver skin was from the thigh of a human male, 79 years old.

TABLE 37
Diclofenac Spray Composition

| | Composition, wt. % | | | | | |
|---|---|---|---|---|---|---|
| Ingredients | DcS03 | DcS13 | DcS13a | DcS13a-2 | DcS15 | DcS15a |
| Diclofenac | 1 | 1 | 1 | 1 | 1 | 1 |
| diethylamine | | | | | | |
| Propylene | 10 | 15 | 10 | 15 | 10 | 10 |
| glycol | | | | | | |
| Lauryl | 3 | 3 | 3 | 3 | 3 | 3 |
| lactate | | | | | | |
| Lactic acid | 1.5 | 1.5 | 1.5 | 1.5 | 0.5 | 0.5 |
| Brij 58¹ | 0 | 3 | 3 | 3 | 3 | 3 |
| Glyceryl | 3 | 3 | 3 | 3 | 3 | 3 |
| monolaurate | | | | | | |
| Ethanol | 48.5 | 23 | 38 | 45 | 26 | 39.5 |
| Water | 33 | 55.5 | 40.5 | 28.5 | 53.5 | 40 |
| TOTAL | 100 | 105 | 100 | 100 | 100 | 100 |

Compositions DcS13, DcS13a and DCS15 were cloudy.

TABLE 38
Diclofenac Permeation Data

| | Cumulative Amount in Receptor, μg/cm² | | | | | | | |
|---|---|---|---|---|---|---|---|---|
| Time, hours | DcS03 | ±SD | DcS13a-2 | ±SD | DcS15a | ±SD | Voltaren 1% | ±SD |
| 2 | 5.99 | 2.58 | 4.84 | 3.56 | 3.42 | 0.79 | 0.00 | 0.00 |
| 4 | 14.39 | 6.54 | 11.65 | 5.16 | 10.00 | 1.87 | 3.36 | 1.11 |
| 6 | 21.60 | 9.52 | 17.61 | 6.29 | 16.14 | 3.04 | 7.10 | 1.33 |

Formulations DcS13, DcS13a, and DcS15 were cloudy. The above data show that incorporation of Brij 58 reduced skin permeation of diclofenac.

The effects of propylene glycol and thickeners on ketoprofen skin permeation were investigated. The experimental results obtained with a ketoprofen spray formulation using ketoprofen, different levels of propylene glycol, and thickeners hydroxypropyl cellulose (100 cps) and hydroxypropyl cellulose (150-400 cps) are presented in Tables 39 and 40, below and in FIG. 20. The experimental procedure was the same as that for the previous spray compositions, except that the dermatomed cadaver skin was from the thigh of a human male, 79 years old.

TABLE 39
Ketoprofen and Thickeners Spray Composition

| | Composition, wt. % | | | | |
|---|---|---|---|---|---|
| Ingredients | KeS47 | KeS84 | KeS85 | KeS86 | KeS89 |
| Ketoprofen | 5 | 5 | 5 | 5 | 5 |
| Propylene glycol | 10 | 20 | 20 | 10 | 10 |
| Lauryl lactate | 3 | 3 | 3 | 3 | 3 |
| Lactic acid | 1.5 | 1.5 | 1.5 | 1.5 | 1.5 |
| HPC HY117¹² | 0 | 0 | 0.5 | 0.5 | 0 |
| HPC HY119¹³ | 0 | 0 | 0 | 0 | 0.25 |
| Glyceryl monolaurate | 3 | 3 | 3 | 3 | 3 |
| Ethanol | 37.5 | 27.5 | 27.5 | 32.5 | 32.25 |
| Water | 40 | 40 | 39.5 | 44.5 | 45 |
| TOTAL | 100 | 100 | 100 | 100 | 100 |

¹²hydroxypropyl cellulose (100 cps)
¹³hydroxypropyl cellulose (150-400 cps)

TABLE 40
Permeation Data

| | Cumulative Amount in Receptor, μg/cm² | | | | | | | | | | | |
|---|---|---|---|---|---|---|---|---|---|---|---|---|
| Time, | | | | | | | | | | | Profenid | |
| hours | KeS47 | ±SD | KeS84 | ±SD | KeS85 | ±SD | KeS86 | ±SD | KeS89 | ±SD | 2.5% | ±SD |
| 2 | 82.63 | 27.60 | 94.36 | 51.62 | 104.05 | 13.26 | 80.82 | 22.75 | 117.71 | 21.55 | 12.75 | 3.00 |
| 4 | 167.45 | 34.34 | 221.64 | 118.40 | 213.69 | 37.65 | 157.90 | 42.69 | 221.11 | 6.86 | 42.43 | 5.90 |
| 6 | 232.80 | 42.90 | 309.28 | 144.22 | 311.38 | 48.99 | 214.48 | 57.66 | 290.61 | 25.07 | 71.36 | 8.79 |

All formulations above gave clear solutions. The above data show that KeS84, KeS85, and KeS89 all exhibited significant permeation enhancement compared to KeS47. KeS86, with lower propylene glycol, showed similar permeation to KeS47.

The foregoing discussion and the examples are to be taken as illustrative, but not limiting. Still other variants within the spirit and scope of the invention are possible, and will readily present themselves to those skilled in the art.

1. A sprayable solution which comprises a nonsteroidal anti-inflammatory drug (NSAID); lauryl lactate; lactic acid; glyceryl monolaurate; ethanol; and water.

2. The sprayable solution in accordance with claim 1 wherein the NSAID is a propionic acid derivative.

3. The sprayable solution in accordance with claim 1 wherein the NSAID is a propionic acid derivative selected from the group consisting of ketoprofen, ibuprofen, naproxen, and pharmaceutically acceptable salts thereof.

4. The sprayable solution in accordance with claim 1 wherein the NSAID is an acetic acid derivative.

5. The sprayable solution in accordance with claim 1 wherein the NSAID is an acetic acid derivative selected from the group consisting of diclofenac, indomethacin, etodolac, and pharmaceutically acceptable salts thereof.

6. The sprayable solution in accordance with claim 1 further comprising a non-ionic surfactant having an HLB value of at least 12.

7. The sprayable solution in accordance with claim 6 wherein the non-ionic surfactant is an alkoxylated alcohol.

8. The sprayable solution in accordance with claim 1 wherein the NSAID is ketoprofen.

9. The sprayable solution in accordance with claim 1 wherein the water:ethanol weight ratio is in the range of about 0.3:1 to about 2.6:1.

10. The sprayable solution in accordance with claim 1 wherein the lauryl lactate is present in an amount in the range of about 1 to about 5 weight percent, based on the total weight of the solution.

11. A sprayable clear solution which comprises, based on total weight of the solution, a nonsteroidal anti-inflammatory drug (NSAID) in an amount in the range of about 1 to about 10 weight percent,
lauryl lactate in an amount in the range of about 1 to about 5 weight percent,
lactic acid in an amount in the range of about 0.5 to about 5 weight percent;
glyceryl monolaurate in an amount in the range of about 2 to about 5 weight percent;
propylene glycol in an amount in the range of about 5 to about 30 weight percent;
alkoxylated alcohol having a HLB value of at least 12 in an amount in the range of 0 to about 7 percent; and
the remainder a mixture of water and ethanol in a respective weight ratio in the range of about 0.3:1 to about2.6:1.

12. The sprayable clear solution in accordance with claim 11 which comprises about 5 weight percent ketoprofen;
about 3 weight percent lauryl lactate;
about 1.5 weight percent lactic acid;
about 3 weight percent glyceryl monolaurate;
about 3 weight percent polyethylene glycol ether of cetyl alcohol having a HLB value of about 15.7 and represented by the formula CH₃(CH₂)₁₄CH₂(OCH₂CH₂)ₙOH where n has an average value 20;
about 10 weight percent propylene glycol; and the remainder a water-ethanol mixture in a respective weight ratio of about 1.7.
